(19) United States
(12) Patent Application Publication
(10) Pub. No.: US 2014/0358580 A1
(43) Pub. Date: Dec. 4, 2014

(54) METHOD OF AUTOMATICALLY PROCESSING TEST STRIP PURCHASE NOTICE INFORMATION

(71) Applicant: Philosys Co., Ltd., Gunsan-si, Jeollabuk-do (KR)

(72) Inventors: In Hwan Choi, Suwon-si (KR); Jeog In Bag, Anyang-si (KR)

(21) Appl. No.: 14/366,771

(22) PCT Filed: Dec. 24, 2012
(86) PCT No.: PCT/KR2012/011396

§ 371 Date: Jun. 19, 2014

(30) Foreign Application Priority Data
Jan. 3, 2012 (KR) 10-2012-0000426

(51) Int. Cl. H04M 1/21; A61B 5/15; H04M 1/23; A61B 19/00; G06Q 20/32; G06F 19/00

(52) U.S. Cl. CPC H04M 1/21; G06Q 20/322; G06F 19/36; H04M 1/23; A61B 19/44; A61B 5/150358
USPC 705/2

(57) ABSTRACT

The present invention relates to a method of automatically processing test strip purchase notice information. When a test strip is inserted into a bio-data measuring device, the bio-data measuring device recognizes insertion of the test strip. The bio-data measuring device measures bio-data from a material absorbed by the test strip, and counts the test strip used to measure the bio-data as a validly used test strip. The bio-data measuring device stores a count value obtained by counting the test strip as a validly used test strip. If a resulting count value, obtained by repeating above operations and increasing the count value, reaches a predetermined number, the bio-data measuring device automatically transmits information about the resulting count value to a server. The server receives the information about the resulting count value, and automatically transmits test strip purchase notice information to the bio-data measuring device.

### TECHNICAL FIELD

The present invention relates, in general, to a method of automatically processing test strip purchase notice information and, more particularly, to a method of automatically processing test strip purchase notice information, which can count the number of test strips used and can automatically provide test strip purchase notice information to a user.

### BACKGROUND ART

A test strip refers to test paper or a test film used to absorb a liquid biological sample, such as blood, urine, saliva, or other body fluids, and analyze biological components, such as glucose, protein, ketone body, bilirubin, occult blood, and/or nitrite present in the liquid biological sample. Such a test strip is inserted into and used within a biological sample analysis device.

For example, each diabetic individually purchases a blood glucose monitoring device and a test strip required to measure blood glucose, collects blood, uses the blood glucose measurement test strip to absorb collected blood, inserts the blood glucose measurement test strip containing the collected blood into the blood glucose monitoring device, and checks a blood glucose level. In this procedure, since the conventional blood glucose monitoring device does not have the function of counting the number of test strips, and does not provide a service for information about the purchasing of test strips, a user must check the number of remaining unused test strips each time they use the device. If the monitoring of the number of remaining test strips is not accurately conducted, a case may occur where blood glucose may not be measured due to the absence of a test strip in an emergency situation in which blood glucose must be urgently measured.

Further, test strips are generally sold in packs of 50. Accordingly, when the biological sample analysis device has the function of counting the number of test strips used therefor, providing notice to the user to purchase more test strips when the number of test strips (a count value) reaches a predetermined number, and enabling the purchasing of test strips to be automatically processed, the user can conveniently use the biological sample analysis device without separately managing the purchasing of test strips.

Therefore, the present inventors developed a method of automatically processing test strip purchase notice information, which can count the number of test strips used so that the number of times bio-data is measured can be accurately checked, which can automatically provide test strip purchase notice information to a user, and which enable extra test strips to be immediately purchased.

### DISCLOSURE OF INVENTION

### Technical Problem

Accordingly, the present invention has been made keeping in mind the above problems occurring in the prior art, and an object of the present invention is to provide a method of automatically processing test strip purchase notice information, which can count the number of test strips used so that the number of times bio-data is measured can be accurately checked.

Another object of the present invention is to provide a method of automatically processing test strip purchase notice information, which can automatically provide test strip purchase notice information to a user.

A further object of the present invention is to provide a method of automatically processing test strip purchase notice information, which enables extra test strips to be immediately purchased.

The above and other objects of the present invention can be achieved by the present invention which will be described later.

### Solution to Problem

In accordance with an aspect of the present invention to accomplish the above objects, there is provided a method of automatically processing test strip purchase notice information, including a) when a test strip is inserted into a bio-data measuring device, the bio-data measuring device recognizing insertion of the test strip; b) the bio-data measuring device measuring bio-data from a material absorbed by the test strip, and counting the test strip used to measure the bio-data as a validly used test strip; c) the bio-data measuring device storing a count value obtained by counting the test strip as a validly used test strip; d) if a resulting count value, obtained at c) by repeating a) to c) and increasing the count value, reaches a predetermined number, the bio-data measuring device automatically transmitting information about the resulting count value to a server; and e) the server receiving the information about the resulting count value, and automatically transmitting test strip purchase notice information to the bio-data measuring device.

Preferably, c) may include the bio-data measuring device outputting visual or auditory information about the count value, obtained by counting the test strip as a validly used test strip.

Preferably, e) may include the server providing information about payment of a user to the bio-data measuring device.

Preferably, d) may include automatically transmitting bio-data of a user stored in the bio-data measuring device to the server, and e) may include the server providing data related to the automatically transmitted bio-data of the user to the bio-data measuring device, thus enabling data to be exchanged via a ubiquitous (U)-healthcare system.

Preferably, d) and e) may be configured to exchange data via Near Field Communication (NFC).

In accordance with another aspect of the present invention to accomplish the above objects, there is provided a method of automatically processing test strip purchase notice information, including a) when a test strip is inserted into a bio-data measuring device, the bio-data measuring device recognizing insertion of the test strip; b) the bio-data measuring device measuring bio-data from a material absorbed by the test strip, and counting the test strip used to measure the bio-data as a validly used test strip; c) the bio-data measuring device storing a count value obtained by counting the test strip as a validly used test strip; d) the bio-data measuring device automatically transmitting the count value, obtained at c) by counting the test strip as a validly used test strip, to a server, and the server receiving the count value; and e) if a resulting count value, obtained at d) by repeating a) to d) and increasing the count value, reaches a predetermined number, the server automatically transmitting information about the resulting count value and test strip purchase notice information to the bio-data measuring device.

Preferably, e) may include the server providing information about payment of a user to the bio-data measuring device.

Preferably, d) may include automatically transmitting bio-data of a user stored in the bio-data measuring device to the server, and e) may include the server providing data related to the automatically transmitted bio-data of the user to the bio-data measuring device, thus enabling data to be exchanged via a ubiquitous (U)-healthcare system.

Preferably, d) and e) may be configured to exchange data via Near Field Communication (NFC).

In accordance with a further aspect of the present invention to accomplish the above objects, there is provided a method of automatically processing test strip purchase notice information, including a) when a test strip is inserted into a blood glucose measurement module, the blood glucose measurement module recognizing insertion of the test strip; b) the blood glucose measurement module measuring bio-data from a material absorbed by the test strip, and counting the test strip used to measure the bio-data as a validly used test strip; c) transmitting information about a count value, obtained by counting the test strip as a validly used test strip, to a smart phone, and the smart phone storing the information about the count value obtained by counting the test strip as a validly used test strip; d) if a resulting count value, obtained at c) by repeating a) to c) and increasing the count value, reaches a predetermined number, the smart phone automatically transmitting information about the resulting count value to a server; and e) the server receiving the information about the resulting count value, and automatically transmitting test strip purchase notice information to the smart phone, wherein the blood glucose measurement module includes a main body including a blood glucose measurement unit for measuring blood glucose contents in blood absorbed by the test strip, and a central processing unit for calculating and transmitting results of measurement of the blood glucose contents and a count value obtained by counting validly used test strips; a test strip insertion slot formed in a portion of the main body and configured to have a structure enabling the test strip to be inserted into the test strip insertion slot; and a plug formed on one side surface of the main body and configured to have a structure that can be coupled to a socket of the smart phone, the plug being coupled to the socket of the smart phone.

Preferably, the smart phone may include an application program capable of processing and managing the results of the measurement of the blood glucose contents and the count value obtained by counting the validly used test strips.

Preferably, c) may include the smart phone outputting visual or auditory information about the count value obtained by counting the validly used test strips.

Preferably, e) may include the server providing information about payment of a user to the smart phone.

Preferably, d) may include automatically transmitting bio-data of a user stored in the smart phone to the server, and e) may include the server providing data related to the automatically transmitted bio-data of the user to the smart phone, thus enabling data to be exchanged via a ubiquitous (U)-healthcare system.

Preferably, d) and e) may be configured to exchange data via Near Field Communication (NFC).

In accordance with yet another aspect of the present invention to accomplish the above objects, there is provided a method of automatically processing test strip purchase notice information, including a) when a test strip is inserted into a blood glucose measurement module, the blood glucose measurement module recognizing insertion of the test strip; b) the blood glucose measurement module measuring bio-data from a material absorbed by the test strip, and counting the test strip used to measure the bio-data as a validly used test strip; c) transmitting information about a count value, obtained by counting the test strip as a validly used test strip, to a smart phone, and the smart phone storing the information about the count value obtained by counting the test strip as a validly used test strip; d) the smart phone automatically transmitting the count value, obtained at c) by counting the test strip as a validly used test strip, to the server, and the server receiving the count value; and e) if a resulting count value, obtained at d) by repeating a) to d) and increasing the count value, reaches a predetermined number, the server automatically transmitting information about the resulting count value and test strip purchase notice information to the smart phone, wherein the blood glucose measurement module includes a main body including a blood glucose measurement unit for measuring blood glucose contents in blood absorbed by the test strip, and a central processing unit for calculating and transmitting results of measurement of the blood glucose contents and a count value obtained by counting validly used test strips; a test strip insertion slot formed in a portion of the main body and configured to have a structure enabling the test strip to be inserted into the test strip insertion slot; and a plug formed on one side surface of the main body and configured to have a structure that can be coupled to a socket of the smart phone, the plug being coupled to the socket of the smart phone.

Preferably, the smart phone may include an application program capable of processing and managing the results of the measurement of the blood glucose contents and the count value obtained by counting the validly used test strips.

Preferably, e) may include the server providing information about payment of a user to the smart phone.

Preferably, d) may include automatically transmitting bio-data of a user stored in the smart phone to the server, and e) may include the server providing data related to the automatically transmitted bio-data of the user to the smart phone, thus enabling data to be exchanged via a ubiquitous (U)-healthcare system.

Preferably, d) and e) may be configured to exchange data via Near Field Communication (NFC).

### Advantageous Effects of Invention

The present invention is advantageous in that it can provide a method of automatically processing test strip purchase notice information, which can count the number of test strips used so that the number of times bio-data is measured can be accurately checked, which can automatically provide test strip purchase notice information to a user, and which enables extra test strips to be immediately purchased.

### BRIEF DESCRIPTION OF DRAWINGS

FIG. 1 is a flowchart schematically showing a method of automatically processing test strip purchase notice information according to an embodiment of the present invention;

FIG. 2 is a flowchart schematically showing a method of automatically processing test strip purchase notice information according to another embodiment of the present invention;

FIG. 3 is a flowchart schematically showing a method of automatically processing test strip purchase notice information according to a further embodiment of the present invention;

FIG. 4 is a flowchart schematically showing a method of automatically processing test strip purchase notice information according to yet another embodiment of the present invention;

FIG. 5 is a perspective view schematically showing the appearance of a blood glucose measurement module according to still another embodiment of the present invention; and

FIG. 6 is a perspective view schematically showing the appearance of a blood glucose measurement module and a smart phone according to still another embodiment of the present invention.

### BEST MODE FOR CARRYING OUT THE INVENTION

The above and other objects, features and advantages of the present invention will be more clearly understood from the following detailed description taken in conjunction with the accompanying drawings. Hereinafter, a method of automatically processing test strip purchase notice information according to embodiments of the present invention will be described in detail with reference to the attached drawings.

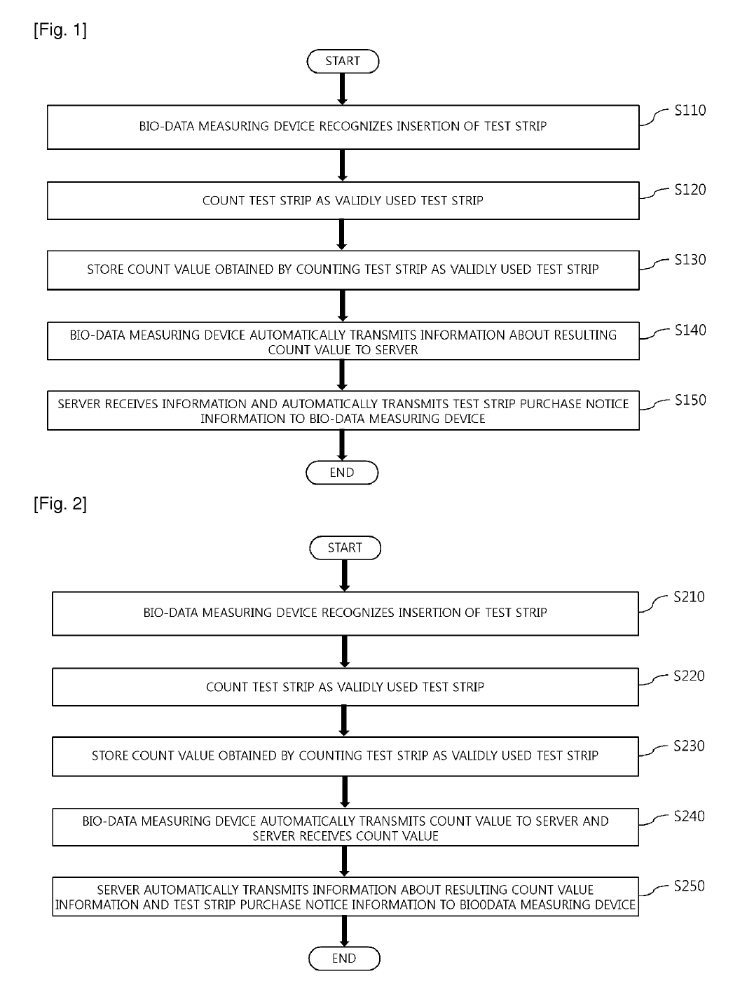

FIG. 1 is a flowchart schematically showing a method of automatically processing test strip purchase notice information according to an embodiment of the present invention.

As shown in FIG. 1, a method of automatically processing test strip purchase notice information according to an embodiment of the present invention includes steps S110 to S150. At step S110, when a test strip is inserted into a bio-data measuring device, the bio-data measuring device recognizes the insertion of the test strip. At step S120, the bio-data measuring device measures bio-data from a material absorbed by the test strip, and counts the test strip used to measure the bio-data as a validly used test strip. At step S130, the bio-data measuring device stores a count value obtained by counting the corresponding test strip as a validly used test strip. If a resulting count value, obtained at step S130 by repeating steps S110 to S130 and increasing the count value, reaches a predetermined number, the bio-data measuring device automatically transmits information about the resulting count value to a server at step S140. At step S150, the server receives information about the resulting count value, and automatically transmits test strip purchase notice information to the bio-data measuring device.

Step S110 is the step of the bio-data measuring device recognizing the insertion of the test strip. The user may insert a test strip into the bio-data measuring device without conducting the action of causing a material for bio-data measurement to be absorbed by the test strip, and then reuse the inserted test strip. In this case, the bio-data measuring device does not count the corresponding test strip as a validly used test strip. Step S120 corresponds to a case where a material for bio-data measurement is absorbed by the test strip. In this case, the bio-data measuring device measures bio-data from the material absorbed by the test strip, and counts the test strip used to measure the bio-data as a validly used test strip. That is, the bio-data measuring device measures bio-data from the material absorbed by the test strip, and the user would not reuse the test strip by which the material for bio-data measurement has already been absorbed. Accordingly, in this case, the bio-data measuring device regards the used test strip as a validly used test strip, and increases a count value to indicate that one test strip has been used. Thereafter, the bio-data measuring device performs the step S130 of storing the count value obtained by counting the corresponding test strip as a validly used test strip.

Thereafter, if the resulting count value, obtained at step S130 by repeating steps S110 to S130 and increasing the count value, reaches a predetermined number, the step S140 of the bio-data measuring device automatically transmitting information about the resulting count value to the server is performed. For example, the bio-data measuring device repeats the operation of increasing the count value to indicate that one test strip has been used, for the validly used test strip. If the resulting count value reaches a predetermined number, the bio-data measuring device automatically transmits information about the resulting count value to the server. Generally, since test strips are sold in packs of 50, it is preferable to perform setting such that if the resulting count value reaches a specific one of numbers ranging from 40 to 45, the bio-data measuring device automatically transmits information about the resulting count value to the server. After step S140, the server that received the information about the resulting count value performs the step S150 of automatically transmitting test strip purchase notice information to the bio-data measuring device.

Step S130 may be configured to include the step of the bio-data measuring device outputting visual or auditory information about the count value obtained by counting the corresponding test strip as a validly used test strip. In this case, the output of visual information is preferably configured to display the visual information using a display component provided in the bio-data measuring device, and the output of auditory information is preferably configured to output a beep sound implemented in the bio-data measuring device to the outside of the device.

Step S150 may be configured to include the step of the server providing information about the payment of a consumer (user) to the bio-data measuring device. When the server provides information about the payment of the consumer, together with the test strip purchase notice information, to the bio-data measuring device, there is an advantage in that the consumer can check useful test strip purchase notice information, pay for test strips, and then conveniently purchase the test strips.

The method of automatically processing test strip purchase notice information according to the present invention can be implemented using various communication methods. For example, when the test strip purchase notice information is automatically processed by a Ubiquitous (U)-healthcare system, the bio-data measuring device can provide various types of bio-data measurement values, measured by the bio-data measuring device, as well as information about the resulting count value, to a plurality of servers provided in a pharmacy, a hospital, a healthcare corporation, etc., receive data related to the bio-data of the user in real time from the plurality of servers, and monitor the data. Also, the bio-data measuring device can be connected to a hospital and a doctor over a network and can be provided with medical examination and treatment information. Furthermore, steps S140 and S150 can be configured to exchange data via Near Field Communication (NFC), such as Bluetooth.

FIG. 2 is a flowchart schematically showing a method of automatically processing test strip purchase notice information according to another embodiment of the present invention.

As shown in FIG. 2, the method of automatically processing test strip purchase notice information according to another embodiment of the present invention includes steps S210 to S250. At step S210, when a test strip is inserted into a bio-data measuring device, the bio-data measuring device recognizes the insertion of the test strip. At step S220, the bio-data measuring device measures bio-data from a material absorbed by the test strip, and counts the test strip used to measure the bio-data as a validly used test strip. At step S230, the bio-data measuring device stores a count value obtained by counting the corresponding test strip as a validly used test strip. At step S240, the bio-data measuring device automatically transmits the count value, obtained at step S230 by counting the test strip as a validly used test strip, to the server, and the server receives the count value. If a resulting count value, obtained at step S240 by repeating steps S210 to S240 and increasing the count value, reaches a predetermined number, the server automatically transmits information about the resulting count value and test strip purchase notice information to the bio-data measuring device at step S250.

Steps S210 to S230 are performed in the same manner as that of steps S110 to S130 of FIG. 1. However, step S240 is the step of the bio-data measuring device automatically transmitting the increased count value obtained by counting all validly used test strips to the server, and the server receiving the count value, wherein the resulting count value is processed by the server. Therefore, if the resulting count value, obtained by repeating steps S210 to S240 and increasing the count value, reaches a predetermined number, the server automatically transmits information about the resulting count value and test strip purchase notice information to the bio-data measuring device at step S250.

Step S250 may be configured to include the step of the server providing information about the payment of a consumer to the bio-data measuring device. When step S240 includes the step of automatically transmitting the bio-data of the user stored in the bio-data measuring device to the server, and step S250 further includes the step of providing data related to the automatically transmitted bio-data of the user to the bio-data measuring device, the exchange of data is performed by the U-healthcare system, thus obtaining the advantages of acquiring various types of health information and enabling remote treatment to be performed in real time. Further, steps S240 and S250 may be configured to exchange data via NFC, such as Bluetooth.

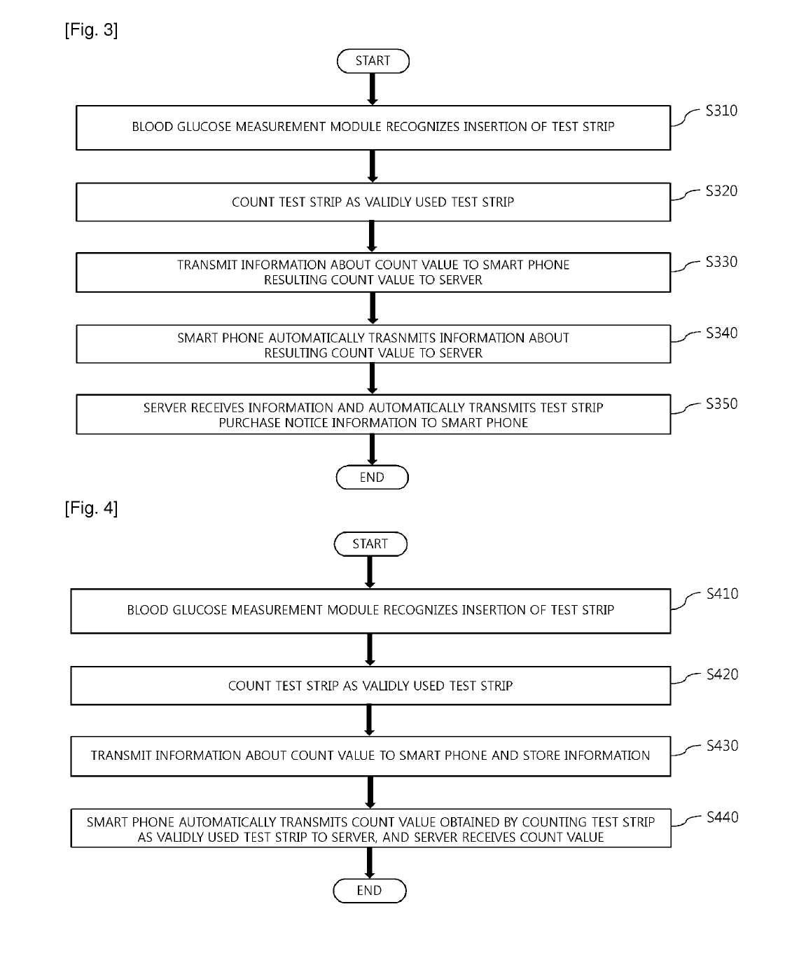

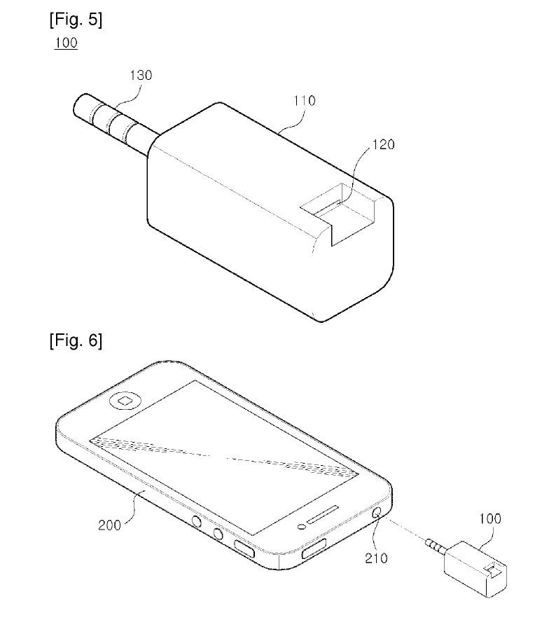

FIGS. 3 and 4 are flowcharts schematically showing a method of automatically processing test strip purchase notice information according to other embodiments of the present invention, FIG. 5 is a perspective view schematically showing the appearance of a blood glucose measurement module according to still another embodiment of the present invention, and FIG. 6 is a perspective view schematically showing the appearance of a blood glucose measurement module and a smart phone according to still another embodiment of the present invention.

As shown in FIGS. 3 to 6, a method of automatically processing test strip purchase notice information according to a further embodiment of the present invention includes steps S310 to S350. At step S310, when a test strip is inserted into a blood glucose measurement module, the blood glucose measurement module recognizes the insertion of the test strip. At step S320, the blood glucose measurement module measures bio-data from a material absorbed by the test strip, and counts the test strip used to measure the bio-data as a validly used test strip. At step S330, information about a count value obtained by counting the test strip as a validly used test strip is transmitted to a smart phone, and the smart phone stores the information about the count value obtained by counting the test strip as a validly used test strip. If a resulting count value, obtained at step S330 by repeating steps S310 to S330 and increasing the count value, reaches a predetermined number, the smart phone automatically transmits information about the resulting count value to a server at step S340. At step S350, the server receives information about the resulting count value, and automatically transmits test strip purchase notice information to the smart phone.

Step S310 is the step of, when the test strip is inserted into the blood glucose measurement module, the blood glucose measurement module recognizing the insertion of the test strip. Since the blood glucose measurement module includes a blood glucose measurement unit and a central processing unit, it is configured to recognize whether the test strip has been inserted. Thereafter, the blood glucose measurement module performs the step S320 of measuring bio-data from the material absorbed by the test strip, determining that the test strip used to measure the bio-data is a validly used test strip, and then increasing a count value to indicate that one test strip has been used. If it is not determined that the corresponding test strip is a validly used test strip, the process described in the above embodiment of the present invention is performed. Thereafter, step S330 is performed where the information about the count value obtained by counting the test strip as a validly used test strip is transmitted to the smart phone, and the smart phone stores information about the count value obtained by counting the test strip as a validly used test strip.

Thereafter, the step S340 of, if the resulting count value, obtained at step S330 by repeating steps S310 to S330 and increasing the count value, reaches a predetermined number, the smart phone automatically transmitting information about the resulting count value to the server, and the step S350 of the server receiving the information about the resulting count value and automatically transmitting test strip purchase notice information to the smart phone, are sequentially performed. Steps S340 and S350 are performed in the same manner as that of the above embodiment of the present invention.

Furthermore, a method of automatically processing test strip purchase notice information according to yet another embodiment of the present invention includes steps S410 to S450. At step S410, when a test strip is inserted into a blood glucose measurement module, the blood glucose measurement module recognizes the insertion of the test strip. At step S420, the blood glucose measurement module measures bio-data from a material absorbed by the test strip, and counts the test strip used to measure the bio-data, as a validly used test strip. At step S430, information about a count value obtained by counting the test strip as a validly used test strip is transmitted to a smart phone, and the smart phone stores information about the count value obtained by counting the test strip as a validly used test strip. At step S440, the smart phone automatically transmits the count value obtained by counting the test strip as a validly used test strip at step S430 to a server, and the server receives the count value. If a resulting count value, obtained at step S440 by repeating steps S410 to S440 and increasing the count value, reaches a predetermined number, the server automatically transmits information about the resulting count value and test strip purchase notice information to the smart phone at step S450.

In this case, steps S410 to S430 are processed in the same manner as steps S310 to S330 of FIG. 3, and the server receives the increased count value obtained by counting each test strip as a validly used test strip whenever each test strip is measured, and automatically transmits information about the resulting count value and test strip purchase notice information to the smart phone at steps S440 and S450.

Further, the smart phone used according to still another embodiment of the present invention is preferably configured to include an application program capable of processing and managing the results of the measurement of blood glucose contents and the count value obtained by counting validly used test strips.

Step S330 may be configured to include the step of the smart phone outputting visual or auditory information about the count value obtained by counting the corresponding test strip as a validly used test strip. The visual or auditory information is configured, as described above. Each of steps S350 and S450 may be configured to include the step of providing information about the payment of each consumer to the smart phone. In this case, the consumer has the advantage of reducing temporal efforts to manage and purchase test strips.

A blood glucose measurement module according to still another embodiment of the present invention is configured to include a main body 110, a test strip insertion slot 120, and a plug 130. The main body 110 includes a blood glucose measurement unit for measuring blood glucose contents in blood absorbed by a test strip, and a central processing unit for calculating and transmitting a count value obtained by counting validly used test strips. The test strip insertion slot 120 is formed in a portion of the main body and has a structure enabling the test strip to be inserted into the test strip insertion slot. The plug 130 is formed on one side surface of the main body and has a structure that can be coupled to the socket 210 of a smart phone 200, the plug 130 being configured to be coupled to the socket of the smart phone.

The main body 110 is a device containing units for measuring blood glucose, such as the blood glucose measurement unit for measuring blood glucose contents in blood absorbed by the test strip and the central processing unit for calculating and transmitting the results of the measurement of the blood glucose contents, and does not need to separately include a power supply unit and a display unit, thus obtaining the advantage of realizing small size and light weight.

The test strip insertion slot 120 is a unit formed in a portion of the main body and having a structure enabling the test strip to be inserted into the slot. The structure and design of the test strip insertion slot 120 are not especially limited as long as the test strip can be easily inserted into the test strip insertion slot.

Further, the plug 130 is a unit formed on one side surface of the main body and having a structure that can be coupled to the socket of the smart phone. Preferably, the plug 130 has a structure that can be easily coupled to the socket of the smart phone. The type and shape of the plug 130 are not especially limited, but the plug 130 may preferably be implemented as a phone jack plug or a pin plug so that it can be easily coupled to the smart phone.

Furthermore, the blood glucose measurement module 100 according to still another embodiment of the present invention can be used with the module 100 coupled to the socket 210 of the smart phone 200. That is, when the glucose measurement module 100 is used with the plug of the blood glucose measurement module 100 coupled to the socket 210 of the smart phone 200, blood glucose data is obtained in such a way that the blood glucose measurement module 100 measures and calculates blood glucose contents in blood absorbed by the test strip using power supplied by the power supply unit of the smart phone 200 and transmits data about the blood glucose contents to the smart phone 200. During this procedure, the method of automatically processing test strip purchase notice information can be easily implemented using the blood glucose measurement application program of the smart phone 200 capable of processing and managing measured blood glucose data.

When the blood glucose measurement module 100 is used with the plug of the blood glucose measurement module 100 coupled to the socket 210 of the smart phone 200, all of input, output, and power can be used, thus obtaining the advantage of realizing very high commercialization. Further, with regard to a communication scheme related to data, a Frequency Shift Keying (FSK) method is preferably used, but the communication scheme of the present invention is not necessarily limited thereto and can be easily modified in various manners depending on the purpose and means of usage.

The type and shape of the socket 210 are not especially limited, but it is preferable to implement the socket 210 as a phone jack socket that can be coupled to the phone jack plug of the blood glucose measurement module 100 or a port that can be coupled to the pin plug. However, since a coupling structure using the pin can be inversely configured between the smart phone and the blood glucose measurement module, the structure is not necessarily limited to the above-described coupling structure, and it should be understood that structures that can be easily replaced and modified by those skilled in the art belong to the scope of the present invention without departing from the technical spirit of the present invention.

Further, as described above, the method of automatically processing test strip purchase notice information using the blood glucose measurement module and the smart phone can also be performed by means of a U-healthcare system or NFC, such as Bluetooth, through the medium of the smart phone and the server.

Although the preferred embodiments of the present invention have been disclosed for illustrative purposes, those skilled in the art will appreciate that various modifications, additions and substitutions are possible, without departing from the scope and spirit of the invention. Therefore, the scope of the present invention should be defined by the technical spirit of the accompanying claims.

### INDUSTRIAL APPLICABILITY

As described above, the present invention is advantageous in that a consumer can check useful test strip purchase notice information, pay for test strips, and conveniently purchase test strips by using the method of automatically processing test strip purchase notice information according to the present invention.

1. A method of automatically processing test strip purchase notice information, comprising:
a) when a test strip is inserted into a bio-data measuring device, the bio-data measuring device recognizing insertion of the test strip;
b) the bio-data measuring device measuring bio-data from a material absorbed by the test strip, and counting the test strip used to measure the bio-data as a validly used test strip;
c) the bio-data measuring device storing a count value obtained by counting the test strip as a validly used test strip;
d) if a resulting count value, obtained at c) by repeating a) to c) and increasing the count value, reaches a predetermined number, the bio-data measuring device automatically transmitting information about the resulting count value to a server; and
e) the server receiving the information about the resulting count value, and automatically transmitting test strip purchase notice information to the bio-data measuring device.

2. The method of claim 1, wherein c) comprises the bio-data measuring device outputting visual or auditory information about the count value, obtained by counting the test strip as a validly used test strip.

3. The method of claim 1, wherein e) comprises the server providing information about payment of a user to the bio-data measuring device.

4. The method of claim 1, wherein:
d) comprises automatically transmitting bio-data of a user stored in the bio-data measuring device to the server, and e) comprises the server providing data related to the automatically transmitted bio-data of the user to the bio-data measuring device, thus enabling data to be exchanged via a ubiquitous (U)-healthcare system.

5. The method of claim 1, wherein d) and e) are configured to exchange data via Near Field Communication (NFC).

6. A method of automatically processing test strip purchase notice information, comprising:
a) when a test strip is inserted into a bio-data measuring device, the bio-data measuring device recognizing insertion of the test strip;
b) the bio-data measuring device measuring bio-data from a material absorbed by the test strip, and counting the test strip used to measure the bio-data as a validly used test strip;
c) the bio-data measuring device storing a count value obtained by counting the test strip as a validly used test strip;
d) the bio-data measuring device automatically transmitting the count value, obtained at c) by counting the test strip as a validly used test strip, to a server, and the server receiving the count value; and
e) if a resulting count value, obtained at d) by repeating a) to d) and increasing the count value, reaches a predetermined number, the server automatically transmitting information about the resulting count value and test strip purchase notice information to the bio-data measuring device.

7. The method of claim 6, wherein e) comprises the server providing information about payment of a user to the bio-data measuring device.

8. The method of claim 6, wherein d) comprises automatically transmitting bio-data of a user stored in the bio-data measuring device to the server, and e) comprises the server providing data related to the automatically transmitted bio-data of the user to the bio-data measuring device, thus enabling data to be exchanged via a ubiquitous (U)-healthcare system.

9. The method of claim 6, wherein d) and e) are configured to exchange data via Near Field Communication (NFC).

10. A method of automatically processing test strip purchase notice information, comprising:
a) when a test strip is inserted into a blood glucose measurement module, the blood glucose measurement module recognizing insertion of the test strip;
b) the blood glucose measurement module measuring bio-data from a material absorbed by the test strip, and counting the test strip used to measure the bio-data as a validly used test strip;
c) transmitting information about a count value, obtained by counting the test strip as a validly used test strip, to a smart phone, and the smart phone storing the information about the count value obtained by counting the test strip as a validly used test strip;
d) if a resulting count value, obtained at c) by repeating a) to c) and increasing the count value, reaches a predetermined number, the smart phone automatically transmitting information about the resulting count value to a server; and
e) the server receiving the information about the resulting count value, and automatically transmitting test strip purchase notice information to the smart phone,
wherein the blood glucose measurement module comprises:
a main body including a blood glucose measurement unit for measuring blood glucose contents in blood absorbed by the test strip, and a central processing unit for calculating and transmitting results of measurement of the blood glucose contents and a count value obtained by counting validly used test strips;
a test strip insertion slot formed in a portion of the main body and configured to have a structure enabling the test strip to be inserted into the test strip insertion slot; and
a plug formed on one side surface of the main body and configured to have a structure that can be coupled to a socket of the smart phone, the plug being coupled to the socket of the smart phone.

11. The method of claim 10, wherein the smart phone comprises an application program capable of processing and managing the results of the measurement of the blood glucose contents and the count value obtained by counting the validly used test strips.

12. The method of claim 10, wherein c) comprises the smart phone outputting visual or auditory information about the count value obtained by counting the validly used test strips.

13. The method of claim 10, wherein e) comprises the server providing information about payment of a user to the smart phone.

14. The method of claim 10, wherein d) comprises automatically transmitting bio-data of a user stored in the smart phone to the server, and e) comprises the server providing data related to the automatically transmitted bio-data of the user to the smart phone, thus enabling data to be exchanged via a ubiquitous (U)-healthcare system.

15. The method of claim 10, wherein d) and e) are configured to exchange data via Near Field Communication (NFC).

16. A method of automatically processing test strip purchase notice information, comprising:
a) when a test strip is inserted into a blood glucose measurement module, the blood glucose measurement module recognizing insertion of the test strip;
b) the blood glucose measurement module measuring bio-data from a material absorbed by the test strip, and counting the test strip used to measure the bio-data as a validly used test strip;
c) transmitting information about a count value, obtained by counting the test strip as a validly used test strip, to a smart phone, and the smart phone storing the information about the count value obtained by counting the test strip as a validly used test strip;
d) the smart phone automatically transmitting the count value, obtained at c) by counting the test strip as a validly used test strip, to the server, and the server receiving the count value; and
e) if a resulting count value, obtained at d) by repeating a) to d) and increasing the count value, reaches a predetermined number, the server automatically transmitting information about the resulting count value and test strip purchase notice information to the smart phone,
wherein the blood glucose measurement module comprises:
a main body including a blood glucose measurement unit for measuring blood glucose contents in blood absorbed by the test strip, and a central processing unit for calculating and transmitting results of measurement of the blood glucose contents and a count value obtained by counting validly used test strips;
a test strip insertion slot formed in a portion of the main body and configured to have a structure enabling the test strip to be inserted into the test strip insertion slot; and
a plug formed on one side surface of the main body and configured to have a structure that can be coupled to a socket of the smart phone, the plug being coupled to the socket of the smart phone.

17. The method of claim 16, wherein the smart phone comprises an application program capable of processing and managing the results of the measurement of the blood glucose contents and the count value obtained by counting the validly used test strips.

18. The method of claim 16, wherein e) comprises the server providing information about payment of a user to the smart phone.

19. The method of claim 16, wherein d) comprises automatically transmitting bio-data of a user stored in the smart phone to the server, and e) comprises the server providing data related to the automatically transmitted bio-data of the user to the smart phone, thus enabling data to be exchanged via a ubiquitous (U)-healthcare system.

20. The method of claim 16, wherein d) and e) are configured to exchange data via Near Field Communication (NFC).
